(19) United States
(12) Patent Application Publication
(10) Pub. No.: US 2024/0032903 A1
(43) Pub. Date: Feb. 1, 2024

(54) DEVICE FOR MEDICAL SPECIMEN RETRIEVAL

(71) Applicant: Daniel Doerr, Orlando, FL (US)

(72) Inventor: Daniel Doerr, Orlando, FL (US)

(21) Appl. No.: 17/823,580

(22) Filed: Aug. 31, 2022

Related U.S. Application Data
(63) Continuation-in-part of application No. 29/865,461, filed on Jul. 27, 2022.

(51) Int. Cl. A61B 17/00

(52) U.S. Cl. CPC A61B 17/00234; A61B 2017/00287

(57) ABSTRACT

A medical specimen retrieval device with an actuator connected to an introducer tube, a funnel removably attached to an end of the introducer tube, a retracting element extending from an interior of the funnel wherein the biasing retracting element is retractable through the funnel and into an interior of the introducer tube by an actuator, and a medical specimen bag removably coupled to the retracting element.

### CROSS-REFERENCE TO RELATED APPLICATION

This application claims the benefit of priority of U.S. Design application Ser. No. 29/865,461, filed Jul. 27, 2022, the contents of which are herein incorporated by reference.

### BACKGROUND OF THE INVENTION

The present invention relates to medical specimen retrieval and, more particularly, to a device therefor.

Surgical procedures to capture organs or tissue to be removed from a body cavity, such as during laparoscopic surgery via extracorporeal manual morcellation, require devices for removal of medical specimens.

Typically, a mechanism with a medical specimen bag on an end is utilized for retrieval of the medical specimens during such procedures. Those mechanisms enable retraction of the medical specimen bag into the device, normally before use in a medical procedure.

For example, a medical specimen retrieval device may be supplied with the bag deployed. A nurse may retract the bag into an introducer tube of the retrieval device in order for the retrieval device to fit down a working channel of an endoscopic port, typically with an internal diameter between 5 millimeters (mm) to 15 mm. Once the surgeon has inserted the retrieval device through the port, the bag is re-deployed and a specimen is captured. In general, the bag does not re-enter the introducer tube at this point. It is instead released from a retracting element which holds it open. The retracting element is typically a pair of biasing arms but could be a single biasing arm, a continuous wire, or another non-biasing appendage. The retrieval device and bag are then removed separately.

The retraction of the bag into the introducer tube is difficult as the retractor must fight to collapse retracting element of the retrieval device and exhaust air that is trapped in the bag. Currently, the bags are pulled directly into the introducer tube resulting in high and inconsistent force profiles with each pull and potential damage to the tube or to the bag.

The tube is usually made from plastic, which can be deformed by the retracting element retracting into the tube, and excessive deformation may make the device unusable as it must fit into the surgical trocar/cannula of a specific diameter.

In addition, it sometimes takes multiple attempts to retract a bag as the bag is inflated and full of air. The bag does not easily deflate when fitting into the tube. These multiple attempts require extra effort, lead to frustration, and can also cause additional opportunities for damage to the device or bag.

As can be seen, there is a need for a medical specimen retrieval device which does not require such extraneous efforts for retracting the medical specimen bag into the tube.

### SUMMARY OF THE INVENTION

In one aspect of the present invention, a medical specimen retrieval device comprises an introducer tube, a funnel removably attached to an end of the introducer tube wherein the funnel has a proximal end and a distal end and the funnel tapers from a smaller cross-section at the proximal end to a larger cross-section at the distal end, a retracting element extending from an interior of the funnel wherein the retracting element is retractable through the funnel and into an interior of the introducer tube by an actuator, and a medical specimen bag removably coupled to the retracting element.

Advantageously, the funnel protects the end of the introducer tube from damage by the retracting element during retraction and assists in deflation of the bag.

These and other features, aspects and advantages of the present invention will become better understood with reference to the following drawings, description, and claims.

### BRIEF DESCRIPTION OF THE DRAWINGS

FIG. 1 is a front perspective view of a funnel affixed to an introducer tube of a medical specimen retrieval device according to an embodiment of the present invention;

FIG. 2 is a detail perspective view of line 2-2 of FIG. 1;

FIG. 3 is a top plan view of the medical specimen retrieval device thereof in a compacted mode of use;

FIG. 4 is a cross-sectional view taken on line 4-4 of FIG. 3 detailing an actuator of the medical specimen retrieval device;

FIG. 5 is a detail cross-sectional view thereof showing a first step of a retraction of a bag into the introducer tube;

FIG. 6 is a detail cross-sectional view thereof showing a second step of a retraction of the bag into the introducer tube;

FIG. 7 is a detail cross-sectional view thereof showing a third step of a retraction of the bag into the introducer tube;

FIG. 8 is a top plan view of the medical specimen retrieval device thereof in an expanded mode of use; and

FIG. 9 is a cross-sectional view taken on line 9-9 of FIG. 8.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims with reference to the drawings.

A general overview of the various features of the invention will be provided, with a detailed description following. Broadly, an embodiment of the present invention provides a medical specimen retrieval device for retraction and/or retrieval of a medical specimen bag into an introducer tube of the device. The device comprises a funnel attached to the introducer tube.

In some embodiments of the present invention, the funnel may be detachably attached to an end of the introducer tube. The funnel may have a first end (a proximal end) and a second end (a distal end) opposite of the proximal end. The proximal end is configured to attach to the introducer tube of the medical specimen retrieval device. The proximal end may by cylindrical or include a cylindrical segment configured to removably attach to the end of the introducer tube. The proximal end may removably attach to the introducer tube by way of a friction fit or a threaded fit. As such, the proximal end may be configured to friction fit or threadedly attach onto the introducer tube. The introducer tube may have an external diameter approximately between 5 millimeters (mm) and 15 mm. The funnel tapers from a smaller cross-section or cross-sectional view at the proximal end to a larger cross-section at the distal end. The distal end may be a non-rectilinear shape.

In some embodiments, the medical specimen retrieval device may include an actuator connected to a retracting element extending from an interior of the introducer tube and/or from an interior of the funnel. The retracting element may be removably attached or coupled with a medical specimen bag.

The retracting element may be a pair of biasing arms. In some embodiments of the present invention, the retracting element may be at least one biasing arm, at least one non-biasing appendage, and/or a wire, such as a metal wire. The retracting element may be any device suitable to removably attach to the bag, retract into the introducer tube with the bag, and release the bag when not retracted into the introducer tube. In some embodiments, the retracting element may enclose or partially enclose a perimeter of the bag to releasably secure to bag. The retracting element may fit into a sleeve on the bag. Alternatively, the retracting element may attach to the bag by way of a clip. The mechanism by which the retracting element releasably secures the bag is not particularly limited.

The actuator may retract the retracting element and the medical specimen bag through the funnel and into an interior of the introducer tube. The mechanism of the actuator is not particularly limited. The actuator may be any device sufficient to retract the retracting element and medical specimen bag into the introducer tube. In some embodiments, the actuator may function by mechanical means. By way of illustration, the actuator may be a push-pull rod, a tether, a pull, a pulley and/or a combination thereof attached to the retracting element. Activation of the actuator, such as a pull of a lever or pulley, may operate the retracting element.

The medical specimen bag may be flexible and plastic. The medical specimen bag may include an easily accessible opening and a closure suture that facilitates closure of the specimen bag after a specimen has been collected. It may allow capture and capture of multiple specimens from a patient during a procedure.

A deployment mechanism, such as the retracting element, for the bag may comprise a push-pull rod extending through the introducer assembly. This may include or be attached to the actuator. The deployment mechanism may enable a deployment of the bag with the retracting element after a surgical insertion, such as through a cannula. For example, the deployment mechanism may enable the retrieval device to shift between a first position (an open position) and a second position (a closed position). Shifting between said positions may pull the retracting element attached to the medical specimen bag into the introducer tube. This pulling may deflate the bag by forcing the bag into the introducer tube. The funnel may assist by channeling air trapped in the bag for deflation. A deflated bag may more easily retract into and fit into the introducer tube. The funnel may also guide the retracting element and the bag into the introducer tube smoothing the transition of the bag between an open and a closed position.

In some embodiments, after the retracting element is retracted into the retrieval device, the funnel may be removed and discarded. The funnel may be disposable and replaceable. The funnel may be intended for a single use or for multiple uses for a single operation or for a single patient. In some embodiments, the present invention may not be repaired, modified, re-processed, or re-sterilized.

In some embodiments, the device may be attached during manufacturing of the product or attachable after manufacturing.

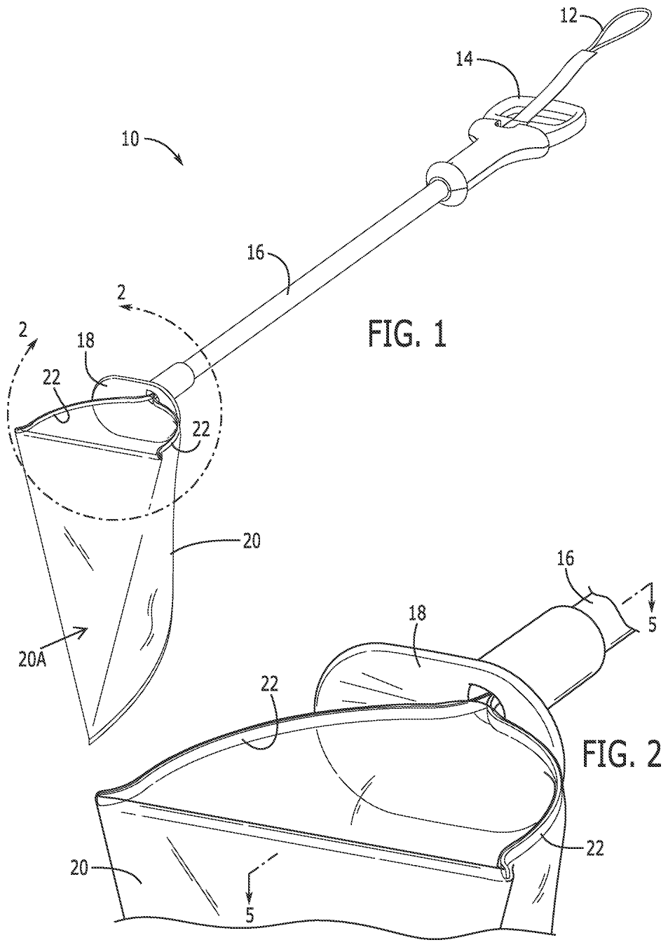

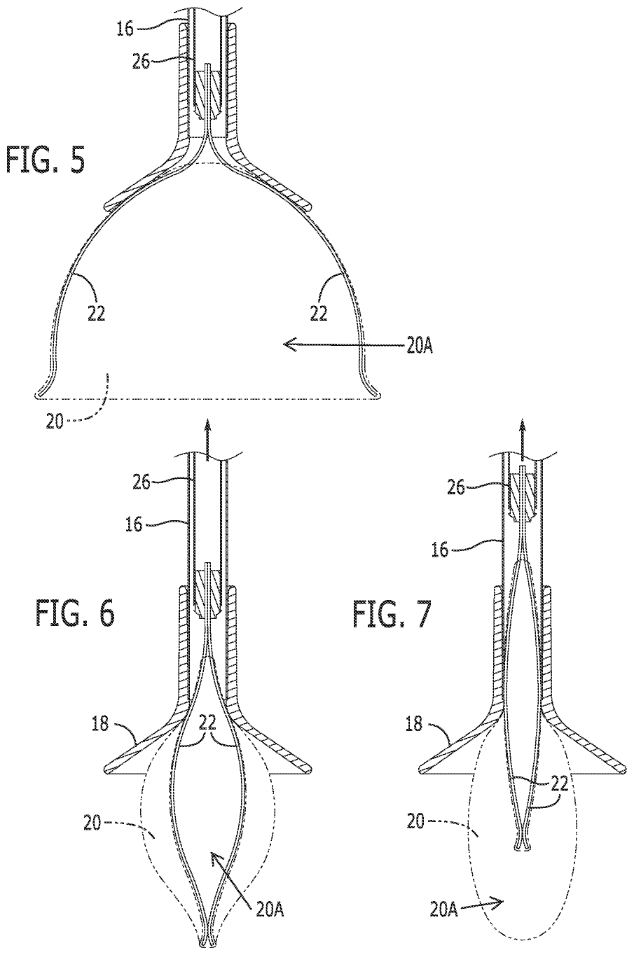

Referring to FIGS. 1 through 9, FIGS. 1 and 2 show a funnel 18 attached to a medical specimen retrieval device 10. The medical specimen retrieval device 10 comprises a tether 12, a handle 14, an introducer tube 16 and biasing arms 22 holding a bag 20. The bag 20 is full of air 20A. The tether 12 functions in combination with a retractable tube 26 as an actuator, retracting the biasing arms 22 into the introducer tube 16 (as best shown in FIGS. 5 through 7). The biasing arms 22 depicted exemplify a retracting element and are depicted by way of example. The present invention may not be limited to the biasing arms as a retracting element.

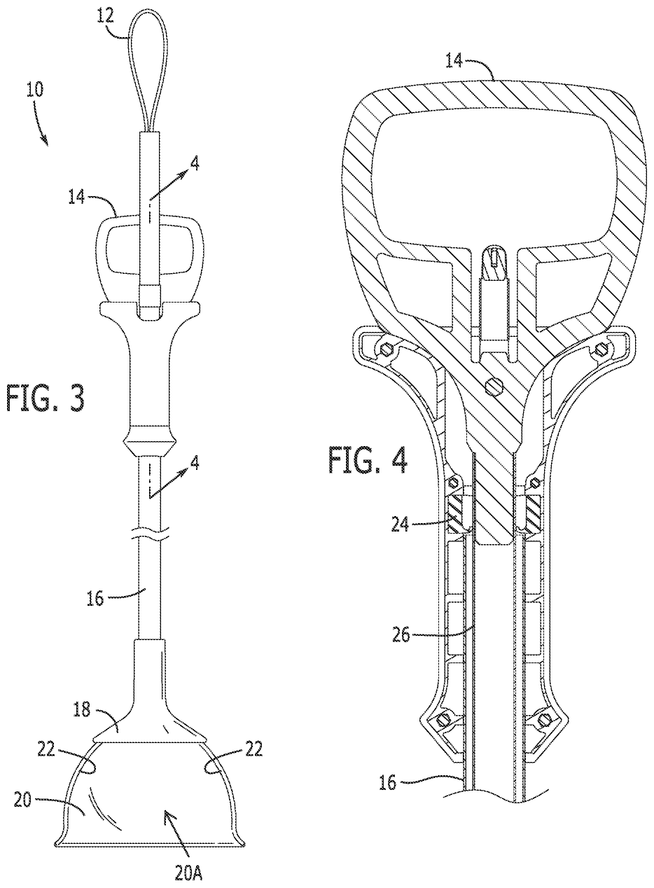

FIGS. 3 and 4 show the medical specimen retrieval device 10 in a compacted state with a retractable tube 26 inside of the introducer tube 16. A seal 24 may be located at an end of the retractable tube 26. In the compacted state, the biasing arms 22 and the bag 20 are fully deployed.

FIGS. 5 through 7 detail steps for retracting the bag 20 into the introducer tube 16 by a retraction of the biasing arms 22. The funnel 18 guides the biasing arms 22 and the bag 20 while simultaneously enabling a route for air 20A to escape from the bag 20 between the biasing arms 22. This enables a smooth retraction of the bag 20 into the introducer tube 16. This retraction is actuated by the tether 12 pulling on the retractable tube 26.

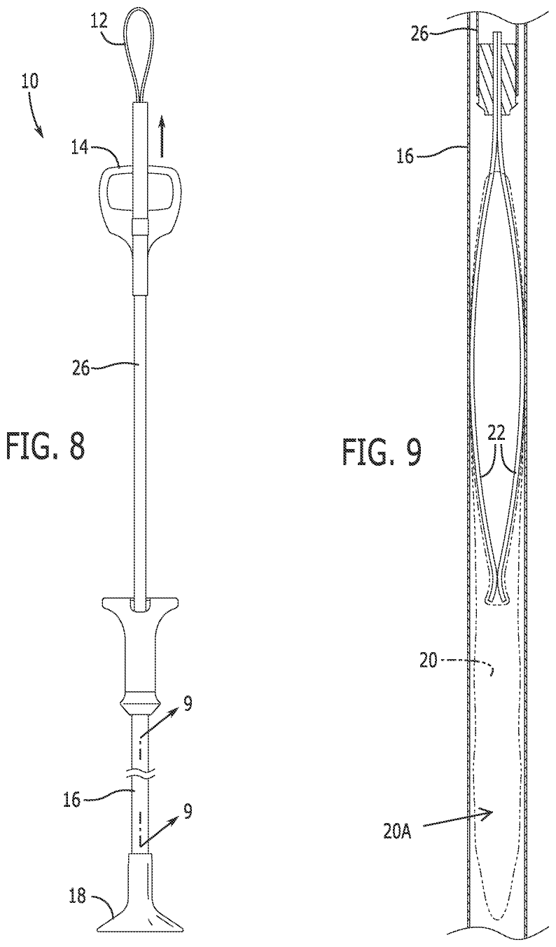

FIG. 8 shows the medical specimen retrieval device 10 in an expanded form with the retractable tube 26 withdrawn from the introducer tube 16. In the expanded form, the biasing arms 22 and the bag 20 are fully retracted into the introducer tube 16.

FIG. 9 shows a cross-sectional view of FIG. 8, detailing the biasing arms 22 and the bag 20 in the introducer tube 16.

It should be understood, of course, that the foregoing relates to exemplary embodiments of the invention and that modifications may be made without departing from the spirit and scope of the invention as set forth in the following claims.

What is claimed is:

1. A medical specimen retrieval device comprising:
an introducer tube;
a funnel, removably attached to an end of the introducer tube, wherein the funnel has a proximal end and a distal end and the funnel tapers from a smaller cross-section at the proximal end to a larger cross-section at the distal end;
a retracting element extending from an interior of the funnel wherein the retracting element is retractable through the funnel and into an interior of the introducer tube by an actuator; and
a medical specimen bag removably coupled to retracting element.

2. The device of claim 1, wherein the proximal end is cylindrical.

3. The device of claim 1, wherein the proximal end includes a cylindrical segment configured to removably attach to the end of the introducer tube.

4. The device of claim 1, wherein the funnel removably attaches to the end of the introducer tube by a friction fit.

5. The device of claim 4, wherein the proximal end is configured to friction fit on the introducer tube wherein the introducer tube is cylindrical with an external diameter between 5 millimeters to 15 millimeters.

6. The device of claim 1, wherein the distal end is a non-rectilinear shape.

7. The device of claim 1, wherein the retractable element comprises at least one biasing arm.

8. The device of claim 1, wherein the retractable element comprises at least one non-biasing appendage.

9. The device of claim 1, wherein the retractable element comprises a wire.

10. A method for using the device of claim 1 comprising:
retracing the retracting element through the funnel and into the introducer tube;
deflating the medical specimen bag by a retraction of the retracting element by the actuator; and
retracting the bag into the introducer tube.
